(19) United States
(12) Patent Application Publication
(10) Pub. No.: US 2026/0013941 A1
(43) Pub. Date: Jan. 15, 2026

(54) PREDICTING ANATOMICAL DISTENSION BASED ON CASE-SPECIFIC FEATURES FOR PERCUTANEOUS ACCESS PROCEDURES

(71) Applicant: AURIS HEALTH, INC., Santa Clara, CA (US)

(72) Inventors: Vahini Ummalaneni, San Francisco, CA (US); Armand Pierre Georges Ledanois, Paris (FR); Evan Lee Cohen, Woodside, CA (US); Elif Ayvali, Redwood City, CA (US)

(21) Appl. No.: 18/773,351

(22) Filed: Jul. 15, 2024

(51) Int. Cl. A61B 34/10; A61B 17/00; A61B 17/34; A61B 34/30

(52) U.S. Cl. CPC A61B 34/10; A61B 17/00234; A61B 17/34; A61B 34/30; A61B 2017/00017; A61B 2017/00238; A61B 2034/105; A61B 2034/302; A61B 2034/303

(57) ABSTRACT

This disclosure provides methods, devices, and systems for planning medical procedures. The present implementations more specifically relate to techniques for using machine learning to predict distension of an anatomy and recommend a plan for percutaneously accessing a target within the anatomy based on the predicted distension. In some aspects, a recommendation system may extract features from input data representing a mapping of an anatomy and infer, from the extracted features, a location on the anatomy for percutaneous entry based on a machine learning model trained on fluidics simulations that predict anatomical distension in response to irrigation. Example suitable input data may include three-dimensional images of the anatomy, two-dimensional images of the anatomy, and/or sensor data received via sensors disposed on an instrument within the anatomy. The recommendation system may further generate a plan for percutaneously accessing a target within the anatomy via the location inferred from the set of features.

### TECHNICAL FIELD

This disclosure relates generally to medical systems, and specifically to predicting anatomical distension based on case-specific features for percutaneous access procedures.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside a patient's body. For example, to remove urinary stones from the kidney and ureter, a physician can insert a ureteroscope into the urinary tract through the urethra. A ureteroscope includes an endoscope at its distal end configured to enable visualization of the urinary tract. Generally, during a percutaneous access procedure (such as PCNL), the physician (or a technician) drives a needle into the patient, through a target location on the kidney, and uses another medical instrument (which may be in conjunction with the needle) to extract the stone from the kidney via the percutaneous access point.

Some medical procedures utilize saline fluids for irrigation and aspiration (also referred to herein as “fluidics”). As used herein, the term “irrigation” refers to the movement or delivery of fluid into an anatomy and the term “aspiration” refers to the movement or extraction of fluid out of the anatomy. Fluidics can be used for various purposes such as, for example, to achieve distension of the anatomy (such as for endoscopic vision), maintain suitable intrarenal pressures during the procedure (such as to prevent damage to the anatomy), or move around objects (such as urinary stones) within the anatomy. Improper management of irrigation and/or aspiration can adversely affect the health of the patient and/or efficacy of the medical procedure. For example, over-pressurization of the anatomy can result in fractures, tissue breakage, or damage to the anatomy. On the other hand, under-pressurization of an anatomy can result in insufficient anatomical distension that is otherwise needed to visualize and/or access certain parts of the anatomy using various medical instruments. Thus, there is a need to plan percutaneous access procedures based on achievable levels of distension at various parts of an anatomy.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method performed by a medical system. The method includes steps of receiving input data representing a mapping of an anatomy; extracting, from the input data, a plurality of features including a position of a target within the anatomy; inferring, from the plurality of features, a location on the anatomy for percutaneous entry based on a machine learning model trained on fluidics simulations that predict anatomical distension in response to irrigation; and generating a recommendation for percutaneously accessing the target via the location inferred from the plurality of features.

Another innovative aspect of the subject matter of this disclosure can be implemented in a controller for a medical system, including a processing system and a memory. The memory stores instructions that, when executed by the processing system, cause the controller to receive input data representing a mapping of an anatomy; extract, from the input data, a plurality of features including a position of a target within the anatomy; infer, from the plurality of features, a location on the anatomy for percutaneous entry based on a machine learning model trained on fluidics simulations that predict anatomical distension in response to irrigation; and generate a recommendation for percutaneously accessing the target via the location inferred from the plurality of features.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows another example medical system, according to some implementations.

FIG. 3 shows an example medical procedure involving fluidics, according to some implementations.

FIG. 4 shows a block diagram of an example controller for a medical system, according to some implementations.

FIG. 5A shows a graphical interface depicting an example percutaneous access plan, according to some implementations.

FIG. 5B shows a segmentation map depicting an example segmentation of the kidney shown in FIG. 5A.

FIGS. 6A-6D show various example kidney types, according to some implementations.

FIG. 7 shows a block diagram of an example machine learning system, according to some implementations.

FIG. 8 shows a block diagram of an example recommendation system for percutaneous access procedures, according to some implementations.

FIG. 9 shows another block diagram of an example controller for a medical system, according to some implementations.

FIG. 10 shows an illustrative flowchart depicting an example operation for generating a percutaneous access recommendation, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, some medical procedures utilize saline fluids for irrigation and aspiration (also referred to herein as “fluidics”). As used herein, the term “irrigation” refers to the movement or delivery of fluid into an anatomy and the term “aspiration” refers to the movement or extraction of fluid out of the anatomy. Fluidics can be used for various purposes such as, for example, to achieve distension of the anatomy (such as for endoscopic vision), maintain suitable intrarenal pressures during the procedure (such as to prevent damage to the anatomy), or move around objects (such as urinary stones) within the anatomy. Improper management of irrigation and/or aspiration can adversely affect the health of the patient and/or efficacy of the medical procedure. For example, over-pressurization of the anatomy can result in fractures, tissue breakage, or damage to the anatomy. On the other hand, under-pressurization of an anatomy can result in insufficient distension that is otherwise needed to visualize and/or access certain parts of the anatomy using various medical instruments. Thus, some physicians (or technicians) may rely on fluidics simulations to predict the achievable levels of distension at various parts of an anatomy when planning a percutaneous access procedure.

As used herein, the term “fluidics simulation” generally refers to any method, technique, and/or system that models or predicts how fluid will flow inside a given volume (such as a kidney or other anatomy). Example suitable fluidics simulations include velocity maps and pressure maps, among other examples. A velocity map of an anatomy shows streamlines and/or velocities of predicted fluid flow at various regions within the anatomy. A pressure map of an anatomy shows predicted fluid pressures at various regions within the anatomy. Aspects of the present disclosure recognize that portions of an anatomy predicted to have higher fluid volume, velocities, and/or pressures are likely to achieve greater distension compared to portions of the anatomy in which the fluid volume, velocities, and/or pressures are more restricted. Thus, a user (such as a physician or a technician) may perform fluidics simulations on an anatomy to select a location on the anatomy (such as a kidney pole) for inserting a percutaneous access instrument (such as a needle or a catheter). For example, the selected location may be predicted to achieve the greatest amount (or at least a threshold amount) of distension under irrigation.

Fluidics simulations rely on three-dimensional (3D) models of a given volume. A 3D model of an anatomy can be extracted or reconstructed from a 3D image of the anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. During a preoperative phase for some medical procedures, an imaging system may be used to scan or otherwise capture images or video of at least a portion of a patient's anatomy. For example, a CT scanner may be used to acquire tomographic images (also referred to as “tomograms” or “CT scans”) of a patient's kidneys during the preoperative phase for a PCNL procedure. A tomogram is a cross-section or slice of a 3D volume such that multiple tomograms can be stacked or combined to recreate the 3D volume (such as a 3D image of a kidney). Thus, fluidics simulations can be performed on CT scans during a preoperative phase of a medical procedure to inform a user about how to gain percutaneous access to a target within an anatomy (such as based on the achievable levels of anatomical distension).

Aspects of the present disclosure recognize that the planning of a percutaneous access procedure can be at least partially automated using machine learning. Machine learning is a technique for improving the ability of a computer system to perform a certain task. Machine learning generally comprises a training phase and an inferencing phase. During the training phase, a machine learning system is provided with one or more “answers” (also referred to as “ground truth”) and a large volume of raw training data associated with the answers. The machine learning system analyzes the training data to learn a set of rules (also referred to as a “machine learning model”) that can be used to describe each of the answers. During the inferencing phase, the machine learning system may infer answers from new data using the learned set of rules. In some aspects, a machine learning model may be trained to infer a plan for percutaneously accessing a target within an anatomy (also referred to as a “percutaneous access plan”) based, at least in part, on a fluidics simulation performed on the anatomy. For example, a percutaneous access plan may include a location on the anatomy for percutaneous entry (such as a kidney pole through which an instrument is to be inserted percutaneously) and/or a positioning of instruments within the anatomy that is likely to result in a successful procedure.

However, fluidics simulations may not be available for some medical procedures. For example, some medical procedures may rely on lower resolution anatomical imaging and/or mapping techniques (such as pyelograms or sensor data captured from within an anatomy) in lieu of the 3D imaging (such as CT scans) that is otherwise needed for fluidics simulations. Aspects of the present disclosure recognize that various anatomies can be parameterized and separated into classes based on known anatomical features (such as a classification of kidney poles, kidney type, or renal pelvis dimensions, among other examples) which can be further correlated or otherwise associated with fluidics simulations performed on such anatomies. More specifically, such anatomical features can be extracted from anatomical maps having various resolutions (including 3D images, pyelograms, and sensor-based mapping). In some aspects, a machine learning model may be further trained to infer, from a set of features of an anatomy, a fluidics simulation for the anatomy (such as a pressure map) and/or a percutaneous access plan associated with the fluidics simulation (such as a recommended location on the anatomy for percutaneous entry and/or a recommended positioning of instruments within the anatomy).

Various aspects relate generally to systems and techniques for planning medical procedures, and more particularly, to techniques for using machine learning to predict distension of an anatomy and recommend a plan for percutaneously accessing a target within the anatomy based on the predicted anatomical distension. In some aspects, a recommendation system may extract a set of features from input data representing a mapping of an anatomy and infer, from the set of features, a location on the anatomy for percutaneous entry based on a machine learning model trained on fluidics simulations that predict anatomical distension in response to irrigation. Example suitable input data may include, among other examples, 3D images of the anatomy (such as CT scans), two-dimensional (2D) images of the anatomy (such as pyelograms), and sensor data received via one or more sensors disposed on an instrument within the anatomy. The recommendation system may further generate a plan for percutaneously accessing a target within the anatomy via the location inferred from the set of features. In some implementations, the plan may include recommended poses of an instrument configured for direct entry and an instrument configured for percutaneous access. In some other implementations, the plan may include a fluidics simulation of the anatomy.

Particular implementations of the subject matter described in this disclosure can be implemented to realize one or more of the following potential advantages. By training a machine learning model to infer a percutaneous access plan based on fluidics simulations that predict anatomical distension in response to irrigation, aspects of the present disclosure may at least partially automate the planning of percutaneous access procedures on a case-specific basis. In other words, the machine learning model may recommend an optimal location on a patient's anatomy for percutaneous entry and/or an optimal positioning of instruments within the anatomy based on anatomical features that are specific to the patient. Because the machine learning model is trained on fluidics simulations, the optimal location for percutaneous entry and/or the optimal positioning of instruments within the anatomy may coincide with a portion of the anatomy that is predicted to achieve the greatest amount of distension (or at least a threshold amount of distension) in response to irrigation. By further training the machine learning model on anatomical features associated with the fluidics simulations, aspects of the present disclosure can infer percutaneous access plans from anatomical maps having various resolutions (including 3D images, pyelograms, and sensor-based mapping).

Aspects of the present disclosure may be used to perform robotic-assisted medical procedures, such as endoscopic access, percutaneous access, or treatment for a target anatomical site. For example, robotic tools may engage or control one or more medical instruments (such as an endoscope and/or a percutaneous access catheter) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure including, for example, minimally invasive procedures (such as laparoscopy), non-invasive procedures (such as endoscopy), therapeutic procedures, diagnostic procedures, percutaneous procedures, non-percutaneous procedures, or other types of procedures. Endoscopic procedures can include bronchoscopy, ureteroscopy, gastroscopy, nephroscopy, and nephrolithotomy, among other examples.

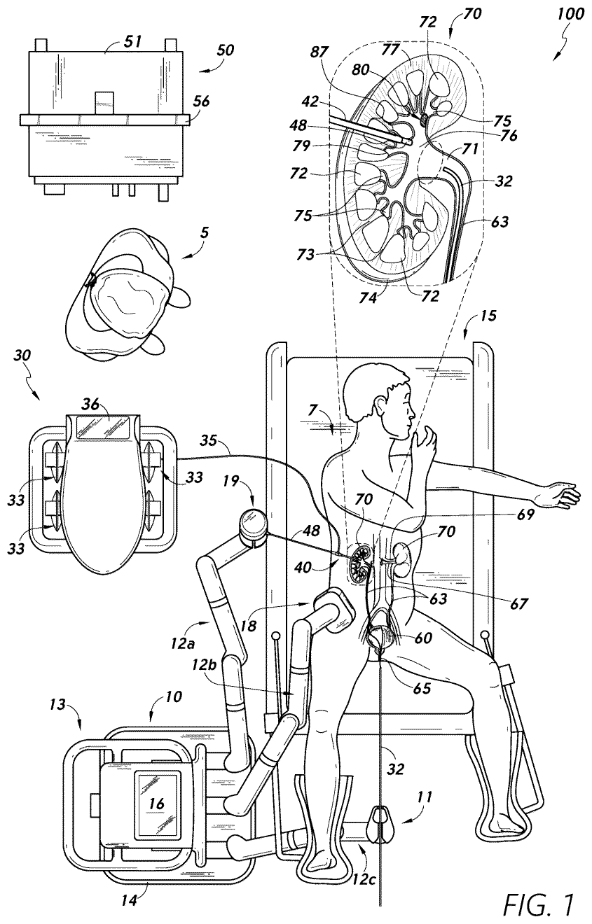

FIG. 1 shows an example medical system 100, according to some implementations. In some aspects, the medical system 100 may be used for percutaneous and/or endoscopic (such as ureteroscopic) medical procedures. As referenced and described above, certain ureteroscopic procedures involve the treatment or removal of kidney stones. In some implementations, kidney stone treatment can benefit from the assistance of certain robotic technologies or devices, such as may be similar to those shown in FIG. 1 and described in detail below. Robotic medical solutions can provide relatively higher precision, superior control, and/or superior hand-eye coordination with respect to certain instruments compared to strictly-manual procedures. For example, robotic-assisted percutaneous access to the kidney in accordance with some procedures can advantageously enable a urologist to perform both direct-entry endoscopic renal access and percutaneous renal access.

In certain stone management procedures, fluid irrigation may be implemented in order to maintain desired kidney distention, which may advantageously facilitate visualization and/or navigation within the target treatment site (such as a calyx networks of a kidney). However, it may be desirable or necessary to limit fluid irrigation at least in part to avoid over-pressurizing the kidney, which can result in physiological harm to the patient and/or damage to the renal anatomy. Specifically, with respect to renal procedures, over-pressurization can result in fractures, tissue breakage, and/or other physical damage. Furthermore, in the presence of active infection, fluid can be dispersed into the bloodstream in response to such breakage, possibly resulting in sepsis, fever, and/or other condition(s). For example, intrarenal infection may result from the presence of one or more kidney stones. Therefore, infected intrarenal fluid that is expelled or otherwise passes into the bloodstream as a result of damage from over-pressurization can result in complications as described above. Therefore, it may be desirable to limit irrigation pressure levels in order to promote the desired or sufficient kidney distention to perform a stone management procedure without causing undesirably high intrarenal pressures. Furthermore, under-pressurization can result in the lack of effective anatomical distention for visualization, which can reduce the efficacy of a procedure and/or result in damage to the internal anatomy.

The medical system 100 includes a robotic system 10 (such as a mobile robotic cart) configured to engage with and/or control a medical instrument 32 (such as a ureteroscope) to perform a direct-entry procedure on a patient 7. The term “direct entry” is used herein according to its broad and ordinary meaning and may refer to any entry of instrumentation through a natural or artificial opening in a patient's body. For example, with reference to FIG. 1, the direct entry of the scope 32 into the urinary tract of the patient 7 may be made via the urethra 65.

In the illustrated system 100, a percutaneous-access instrument 40 is further implemented to provide percutaneous access to the kidney 70. The percutaneous-access instrument 40 may include one or more sheaths and/or shafts through which instruments and/or fluids may access the target anatomy in which the distal end of the instrument 40 is disposed. The term “percutaneous access” is used herein according to its broad and ordinary meaning and may refer to entry, such as by puncture and/or minor incision, of instrumentation through the skin of a patient and any other body layers necessary to reach a target anatomical location associated with a procedure (such as the calyx network of the kidney 70). The term “percutaneous-access instrument” is used herein according to its broad and ordinary meaning and may refer to a surgical tool, device, or assembly that is configured to puncture or to be inserted through skin and/or other tissue or anatomy, such as a needle, a scalpel, a guidewire, sheath, shaft, scope, and the like. However, it should be understood that a percutaneous-access instrument can refer to other types of medical instruments in the context of the present disclosure. Although described in some contexts herein as a nephroscope and an endoscope, respectively, it should be understood that the percutaneous-access instruments 40, 48 and direct-entry instrument 32 may be any type of medical instruments, including endoscopes (such as a ureteroscope), catheters (such as a steerable or non-steerable catheter), a nephroscopes, laparoscopes, or other type of medical instrument.

The system 100 may include a catheter 48, which may access the internal renal anatomy through the percutaneous-access instrument 40. In some implementations, the catheter 48 may be manipulated and/or held in place by a tool or coupling 19 coupled to an arm 12a of the robotic system 10. The catheter 48 may be a flexible, robotically-driven instrument. In some implementations, an aspiration outflow channel may be formed in the space between the outer wall of the catheter 48 and an inner wall or sheath of the percutaneous-access device or assembly 40, wherein the catheter 48 is disposed within a channel formed by such inner wall or sheath. With the catheter 48 disposed within the percutaneous-access instrument 40, the catheter 48 and the shaft(s) or sheath(s) of the percutaneous-access instrument 40 may be generally concentric. The catheter 48 and the percutaneous access instrument 40 may advantageously have generally circular cross-sectional shape over at least a portion thereof.

In some implementations, the percutaneous access instrument or assembly 40 and/or other medical instruments of the system 100 form or provide multiple passive fluid outflow channels. For example, passive outflow channels may include a channel formed between the outer wall of the scope 32 and an access sheath through which the scope 32 is passed or disposed. As another example, a working channel of the scope 32 may provide a passive aspiration outflow path from the kidney 70. In some implementations, active outflow is provided through the percutaneous catheter 48 (such as active suction). In cases in which active suction is not implemented within the percutaneous catheter 48, passive aspiration outflow may flow therethrough to some degree. In some configurations, the greatest volume of passive aspiration outflow may be between the outside of the catheter 48 and the inner wall or sheath of the percutaneous-access instrument or assembly 40.

The medical system 100 also includes a fluid management cart 30, which may be configured to hold one or more fluid bags or containers 33 and/or control fluid flow therefrom. For example, an irrigation fluid line 35 (also referred to as an “irrigation line” or a “fluid line”) may be coupled to one or more of the bags or containers 33 and to an irrigation port of the percutaneous-access instrument or assembly 40. Irrigation fluid may be provided to the target anatomy via the irrigation line 35 and the percutaneous-access instrument or assembly 40. The fluid management cart 30 may include certain electronic components, such as a display 36, flow control mechanics, and/or certain associated control circuitry. The fluid management cart 30 may include a stand-alone tower or cart and may have one or more IV bags 33 hanging on one or more sides thereof. The cart 30 may include one or more pumps with which aspiration fluid may be pulled into a collection container or cartridge. In some implementations, the irrigation fluid pressure may be determined at least in part with respect to one or more points along the irrigation and/or aspiration fluid channel(s).

The medical system 100 also includes a control system 50 configured to interface with the robotic system 10 and/or fluid cart 30, provide information regarding the procedure, and/or perform a variety of other operations. For example, the control system 50 can include one or more display(s) 56 configured to present certain information to assist the physician 5 and/or other technician(s) or individual(s). The medical system 10 can include a table 15 configured to hold the patient 7. The system 10 may further include an electromagnetic (EM) field generator 18, which may be held by one or more of the robotic arms 12 of the robotic system 10 or may be a stand-alone device. Although the various robotic arms are shown in various positions and coupled to various instrumentation, it should be understood that such configurations are shown for convenience and illustration purposes, and such robotic arms may have different configurations over time and/or at different points during a medical procedure. In some implementations, the arm 12a is configured to hold or control the catheter 48 only after removing the electromagnetic field generator 18 therefrom. That is, the instrument coupling 19 and the field generator 18 may generally be mounted to the same robotic arm as interchanged over time.

In some implementations, the system 100 may be used to perform a percutaneous procedure, such as percutaneous nephrolithotomy (PCNL). To illustrate, if the patient 7 has a kidney stone 80 that is too large to be removed or passed through the urinary tract (63, 60, 65), the physician 5 can perform a procedure to remove the kidney stone 80 through a percutaneous access point or path associated with the flank or side of the patient 7. In some implementations, the physician 5 can interact with the control system 50 and/or the robotic system 10 to cause or control the robotic system 10 to advance and navigate the medical instrument 32 (such as a scope) from the urethra 65, through the bladder 60, up the ureter 63, and into the calyx network of the kidney 70 where the stone 80 is located. The physician 5 can further interact with the control system 50 and/or the robotic system 10 to cause or control the advancement of the catheter 48 through the percutaneous-access instrument 40. The control system 50 can provide information via the display(s) 56 that is associated with the medical instrument 32, such as real-time endoscopic images captured therewith, and/or other instruments of the system 100, to assist the physician 5 in navigating or controlling such instrumentation.

The renal anatomy is described herein for reference with respect to certain medical procedures relating to aspects of the present disclosure. The kidneys 70, shown roughly in typical anatomical position in FIG. 1, generally comprise two bean-shaped organs located on the left and right sides, respectively, in the retroperitoneal space. In adult humans, the kidneys are generally about 11 cm m height or length. The kidneys receive blood from the paired renal arteries 69; blood exits the kidney via the paired renal veins 67. Each kidney 70 is fluidly coupled with a respective ureter 63, which generally comprises a tube that carries excreted urine from the kidney 70 to the bladder 60.

A recessed area on the concave border of the kidney 70 is the renal hilum 78, where the renal artery (not shown in the detailed view of the kidney 70) enters the kidney 70 and the renal vein (not shown in detailed view) and ureter 63 leave. The kidney 70 is surrounded by tough fibrous tissue, the renal capsule 74, which is itself surrounded by perirenal fat, renal fascia, and pararenal fat. The anterior (front) surface of these tissues is the peritoneum, while the posterior (rear) surface is the transversalis fascia.

The functional substance, or parenchyma, of the kidney 70 is divided into two major structures: the outer renal cortex 77 and the inner renal medulla 87. These structures take the shape of a plurality of generally cone-shaped renal lobes, each containing renal cortex surrounding a portion of medulla called a renal pyramid 72. Between the renal pyramids 72 are projections of cortex called renal columns 73. Nephrons (not shown in detail in FIG. 1), the urine-producing functional structures of the kidney, span the cortex 77 and medulla 87. The initial filtering portion of a nephron is the renal corpuscle, which is located in the cortex and is followed by a renal tubule that passes from the cortex deep into the medullary pyramids. Part of the renal cortex, a medullary ray, is a collection of renal tubules that drain into a single collecting duct.

The tip or apex, or papilla 79, of each renal pyramid empties urine into a respective minor calyx 75; minor calyces 75 empty into major calyces 76, and major calyces 76 empty into the renal pelvis 71, which transitions to the ureter 63. The manifold-type collection of minor and major calyces may be referred to herein as the “calyx network” of the kidney. At the hilum 78, the ureter 63 and renal vein exit the kidney and the renal artery enters. Hilar fat and lymphatic tissue with lymph nodes surrounds these structures. The hilar fat is contiguous with a fat-filled cavity called the renal sinus. The renal sinus collectively contains the renal pelvis 71 and calyces 75, 76 and separates these structures from the renal medullary tissue. The funnel or tubular-shaped anatomy associated with the calyces can be referred to as the infundibulum or infundibula. That is, an infundibulum generally leads to the termination of a calyx where a papilla is exposed within the calyx.

With further reference to the medical system 100, the medical instrument 32 (such as a scope, directly-entry instrument, etc.) can be advanced into the kidney 70 through the urinary tract. Once at the site of the kidney stone 80 (such as within a target calyx 75 of the kidney 70 through which the stone 80 is accessible), the medical instrument 32 can be used to designate or tag a target location for percutaneous access to the kidney 70. To minimize damage to the kidney and/or surrounding anatomy, the physician 5 can designate a particular papilla 79 of the kidney 70 as the target location or anatomical feature for entering into the kidney 70 with a percutaneous-access instrument (such as a needle). However, other target locations can be designated or determined. Once the percutaneous-access instrument(s) has reached the target location (such as the calyx 75), the utilized percutaneous access path may be used to extract the kidney stone 80 from the patient 7.

Fluid may be directed into the calyx network using the fluid cart 30 and irrigation line 35 throughout at least portions of the procedure to produce desirable kidney distension for navigation and viewing. In cases of under-pressurization, wherein there is not enough fluid in the kidney to produce desired or necessary distension, medical instruments can damage or unintentionally puncture parts of the kidney. For example, laser lithotripsy can result in accidental damage to tissue at the treatment site by the laser as a result of the collapse of the surrounding anatomy from under-pressurization.

In the example of FIG. 1, the medical instrument 32 is implemented as a scope. However, in some other implementations, the medical instrument 32 may be implemented as any suitable type of medical instrument, such as a catheter, a guidewire, a lithotripter, a basket retrieval device, and so on. In some implementations, the medical instrument 32 may be a steerable device.

The various scope-type instruments disclosed herein, such as the scope 32 of the system 100, can be configured to navigate within the human anatomy, such as within a natural orifice or lumen of the human anatomy. The terms “scope” and “endoscope” are used herein according to their broad and ordinary meanings, and may refer to any type of elongate medical instrument having image generating, viewing, and/or capturing functionality and configured to be introduced into any type of organ, cavity, lumen, chamber, or space of a body. A scope can include, for example, a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), colonoscope (such as for accessing the colon and/or rectum), borescope, and so on. Scopes or endoscopes, in some instances, may comprise a rigid or flexible tube, and may be dimensioned to be passed within an outer sheath, catheter, introducer, or other lumen-type device, or may be used without such devices.

Irrigation fluid may be provided to the treatment site (such as the kidney 70) through the percutaneous-access device 40, through the percutaneous-access catheter 48, and/or through the direct-entry device 32. Furthermore, irrigation and aspiration may or may not be provided through the same instrument(s). Where one or more of the instruments (32, 40, 48) provides the irrigation and/or aspiration functionality, one or more others of the instruments may be used for other functionality, such as breaking-up the object 80 to be removed.

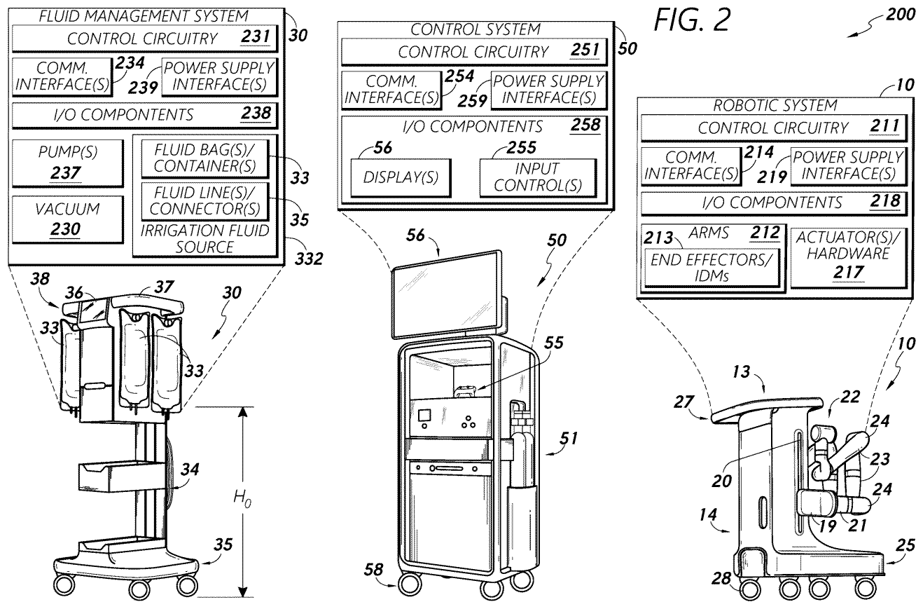

With reference to FIGS. 1 and 2, which shows an example implementation of the control system 50 of FIG. 1 in accordance with one or more implementations, the control system 50 can be configured to provide various functionality to assist in performing a medical procedure. In some implementations, the control system 50 can be coupled to the robotic system 10 and/or fluid management system 30 and operate in cooperation therewith to perform a medical procedure on the patient 7. For example, the control system 50 can communicate with the robotic system 10 and/or fluid management system 30 via a wireless or wired connection (such as to control the robotic system 10, fluid flow from the fluid management system 30, etc.). Further, in some implementations, the control system 50 can communicate with a needle and/or nephroscope to receive position data therefrom. Moreover, in some implementations, the control system 50 can communicate with the table 15 to position the table 15 in a particular orientation or otherwise control the table 15. In some implementations, the control system 50 can communicate with the EM field generator 18 to control generation of an EM field in an area around the patient 7.

FIG. 2 shows another example medical system 200, according to some implementations. In some implementations, the medical system 200 may be one example of the medical system 100 of FIG. 1. For example, the medical system 200 is shown to include the robotic system 10, the control system 50, and the fluid management system 30 of FIG. 1.

The robotic system 10 can be configured to at least partly facilitate execution of a medical procedure. The robotic system 10 can be arranged in a variety of ways depending on the particular procedure. The robotic system 10 can include one or more robotic arms 12 configured to engage with and/or control, for example, the scope 32 and/or a percutaneous access catheter 48 to perform one or more aspects of a procedure. As shown, each robotic arm 12 can include multiple arm segments 23 coupled to joints 24, which can provide multiple degrees of movement or freedom. In the example of FIG. 1, the robotic system 10 is positioned proximate to the patient's legs and the robotic arms 12 are actuated to engage with and position the scope 32 for access into an access opening, such as the urethra 65 of the patient 7. When the robotic system 10 is properly positioned, the scope 32 can be inserted into the patient 7 robotically using the robotic arms 12, manually by the physician 5, or a combination thereof. A scope-advancement instrument coupling 11 (such as an instrument device manipulator) can be attached to the distal portion of one of the robotic arms 12c to facilitate robotic control or advancement of the scope 32. Another one of the robotic arms 12a may have associated therewith an instrument coupling 19 that is configured to facilitate advancement of the percutaneous-access catheter 48. Any of the percutaneous 40, 48 or direct-entry 32 medical instruments may include one or more working channels through which additional tools, such as lithotripters, basket retrieval devices, forceps, etc., can be introduced into the treatment site.

The robotic system 10 can be coupled to any component of the medical system 10, such as to the control system 50, the table 15, the EM field generator 18 (shown coupled to another one of the robotic arms 12b in FIG. 1), the scope 32, and/or any type of percutaneous-access instrument (such as a needle, catheter, or nephroscope). In some implementations, the robotic system 10 is communicatively coupled to the control system 50. For example, the robotic system 10 may be configured to receive a control signal from the control system 50 to perform an operation, such as to position one or more of the robotic arms 12 in a particular manner, manipulate the scope 32, manipulate the catheter 48, and so on. In response, the robotic system 10 can use certain control circuitry 211, actuators 217, and/or other components of the robotic system 10 to perform the operation. In some implementations, the robotic system 10 and/or control system 10 may be configured to receive images and/or image data from the scope 32 representing internal anatomy of the patient 7, such as the urinary system with respect to the particular depiction of FIG. 1, and/or display images based thereon.

With reference to FIG. 2, the robotic system 10 generally includes an elongated support structure 14 (also referred to as a “column”), a robotic system base 25, and a console 13 at the top of the column 14. The column 14 may include one or more arm supports 17 (also referred to as a “carriage”) for supporting the deployment of the one or more robotic arms 12 (three shown in FIG. 2). The arm support 17 may include individually-configurable arm mounts that rotate along a perpendicular axis to adjust the base of the robotic arms 12 for better positioning relative to the patient.

The arm support 17 can be connected to the column 14 through slots 20 that are positioned on opposite sides of the column 14 to guide a vertical translation of the arm support 17. Each of the slots 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, patient sizes, and physician preferences. Similarly, the individually-configurable arm mounts on the arm support 17 can allow a robotic arm base 21 of each of the robotic arms 12 to be angled in a variety of configurations.

The robotic arms 12 may generally comprise robotic arm bases 21 and end effectors 22, separated by a series of linkages 23 that are connected by a series of joints 24, each joint comprising one or more independent actuators 217. Each actuator 217 may comprise an independently-controllable motor. Each independently-controllable joint 24 can provide or represent an independent degree of freedom available to the robotic arm. In some implementations, each of the arms 12 has seven joints, and thus provides seven degrees of freedom, including “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The robotic system base 25 balances the weight of the column 14, arm support 17, and arms 12 over the floor. Accordingly, the robotic system base 25 may house certain relatively heavier components, such as electronics, motors, power supply, as well as components that selectively enable movement or immobilize the robotic system. For example, the robotic system base 25 can include wheel-shaped casters 28 that allow for the robotic system to easily move around the operating room prior to a procedure. After reaching the appropriate position, the casters 28 may be immobilized using wheel locks to hold the robotic system 10 in place during the procedure.

In some implementations, the column 14 may include an input/output (I/O) component 218 configured to receive input, such as user input to control the robotic system 10, and/or provide output, such as a graphical user interface (GUI), information regarding the robotic system 10, or information regarding a procedure, among other examples. The I/O component 218 can include a display, a touchscreen, a touchpad, a projector, a mouse, a keyboard, a microphone, a speaker, etc. In some implementations, the robotic system 10 is movable (such as via the wheel-shaped casters 28) so that the robotic system 10 can be positioned in a location that is appropriate or desired for a procedure. In other implementations, the robotic system 10 may be a stationary system. Further, in some implementations, the robotic system 10 may be integrated into the table 15.

Positioned at the upper end of column 14, the console 13 can provide both a user interface for receiving user input and a display screen 16 (or a dual-purpose device such as, for example, a touchscreen) to provide the physician or user with both pre-operative and intra-operative data. Potential pre-operative data may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 13 may be positioned and/or tilted to allow a physician to access the console from the side of the column 14 opposite arm support 17. From this position, the physician may view the console 13, robotic arms 12, and patient while operating the console 13 from behind the robotic system 10. As shown, the console 13 can also include a handle 27 to assist with maneuvering and stabilizing robotic system 10.

The end effector 22 of each of the robotic arms 12 may comprise an instrument device manipulator (IDM), which may be attached using a mechanism changer interface (MCI). In some implementations, the IDM can be removed and replaced with a different type of IDM, for example, a first type 11 of IDM may manipulate an endoscope, while a second type 19 of IDM may manipulate a catheter. Another type of IDM may be configured to hold an electromagnetic field generator 18. An MCI can include connectors to transfer pneumatic pressure, electrical power, electrical signals, and/or optical signals from the robotic arm 12 to the IDM. The IDMs may be configured to manipulate medical instruments (such as surgical tools or instruments), such as the scope 32, using techniques including, for example, direct drives, harmonic drives, geared drives, belts and pulleys, magnetic drives, and the like. In some implementations, the IDMs can be attached to respective ones of the robotic arms 12, wherein the robotic arms 12 are configured to insert or retract the respective coupled medical instruments into or out of the treatment site. The robotic system 10 further includes power 219 and communication 214 interfaces (such as connectors) to transfer pneumatic pressure, electrical power, electrical signals, and/or optical signals from the robotic arms 12 to the IDMs.

As described with reference to FIG. 1, the medical system 100 can include control circuitry configured to perform certain functionality described herein, including the control circuitry 211 of the robotic system 10, the control circuitry 231 of the fluid management system 30, and/or the control circuitry 251 of the control system 50. That is, the control circuitry of the medical system 100 may be part of the robotic system 10, the fluid management system 30, the control system 50, or some combination thereof. Therefore, any reference herein to control circuitry may refer to circuitry embodied in a robotic system, a fluid management system, a control system, or any other component of a medical system, such as the medical system 100 shown in FIG. 1. The term “control circuitry” is used herein according to its broad and ordinary meaning, and may refer to any collection of processors, processing circuitry, processing modules or units, chips, dies (such as semiconductor dies including come or more active and/or passive devices and/or connectivity circuitry), microprocessors, micro-controllers, digital signal processors, microcomputers, central processing units, field programmable gate arrays, programmable logic devices, state machines (such as hardware state machines), logic circuitry, analog circuitry, digital circuitry, and/or any device that manipulates signals (analog and/or digital) based on hard coding of the circuitry and/or operational instructions.

Control circuitry referenced herein may further include one or more circuit substrates (such as printed circuit boards), conductive traces and vias, and/or mounting pads, connectors, and/or components. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. In implementations where control circuitry comprises a hardware and/or software state machine, analog circuitry, digital circuitry, and/or logic circuitry, data storage device(s) or register(s) storing any associated operational instructions may be embedded within, or external to, the circuitry comprising the state machine, analog circuitry, digital circuitry, and/or logic circuitry.

The control circuitry 211, 231, and/or 251 may comprise a computer-readable medium storing, and/or configured to store, hard-coded and/or operational instructions corresponding to at least some of the steps and/or functions illustrated in one or more of the present figures and/or described herein. Such computer-readable medium can be included in an article of manufacture in some instances. The control circuitry 211, 231, and 251 may be entirely locally maintained or disposed or may be remotely located at least in part (such as communicatively coupled indirectly via a local area network and/or a wide area network). Any of the control circuitry 211, 231, 251 may be configured to perform any aspect(s) of the various processes disclosed herein.

With respect to the robotic system 10, at least a portion of the control circuitry 211 may be integrated with the base 25, column 14, and/or console 13 of the robotic system 10, and/or another system communicatively coupled to the robotic system 10. With respect to the fluid management system 30, at least a portion of the control circuitry 231 may be integrated with a base 31, column 34, and/or console 38 of the fluid management system 30, and/or another system communicatively coupled to the fluid management system 30. With respect to the control system 50, at least a portion of the control circuitry 251 may be integrated with a console base 51 and/or display unit 56 of the control system 50. It should be understood that any description herein of functional control circuitry or associated functionality may be understood to be embodied in the robotic system 10, the fluid management system 30, the control system 50, or any combination thereof, and/or at least in part in one or more other local or remote systems or devices.

With further reference to FIG. 2, the control system 50 can include various I/O components 258 configured to assist the physician 5 or others in performing a medical procedure. For example, the I/O components 258 can be configured to allow for user input to control or navigate the scope 32 and/or catheter 48 within the patient 7. In some implementations, for example, the physician 5 can provide input to the control system 50 and/or robotic system 10, wherein in response to such input, control signals can be sent to the robotic system 10 to manipulate the scope 32 and/or catheter 48. The control system 50 can include one or more display devices 56 to provide various information regarding a procedure. For example, the display(s) 56 can provide information regarding the scope 32 and/or catheter 48. For example, the control system 50 can receive real-time images that are captured by the scope 32 and display the real-time images via the display(s) 56. Additionally, or alternatively, the control system 50 can receive signals (such as analog, digital, electrical, acoustic or sonic, pneumatic, tactile, hydraulic, etc.) from a medical monitor and/or a sensor associated with the patient 7, and the display(s) 56 can present information regarding the health or environment of the patient 7. Such information can include information that is displayed via a medical monitor including, for example, information relating to heart rate (such as ECG or HRV), blood pressure or rate, muscle bio-signals (such as EMG), body temperatures, blood oxygen saturation (such as SpO₂), CO₂, brainwaves (such as EEG), and/or environmental temperatures, among other examples.

To facilitate the functionality of the control system 50, the control system can include various components (sometimes referred to as “subsystems”). For example, the control system 50 can include the control electronics or circuitry 251, as well as one or more power supplies or supply interfaces 259, pneumatic devices, optical sources, actuators, data storage devices, and/or communication interfaces 254. In some implementations, the control system 50 is movable, while in other implementations, the control system 50 is a substantially stationary system. Although various functionality and components are discussed as being implemented by the control system 50, any of such functionality and/or components can be integrated into and/or performed by other systems and/or devices, such as the robotic system 10, the fluid management system 30, the table 15, and/or others, for example. In some implementations, the control system 50 can be coupled to the robotic system 10, the table 15, and/or a medical instrument, such as the scope 32 and/or a needle or other percutaneous-access instrument (not shown), through one or more cables or connections (not shown).

With further reference to FIG. 1, the medical system 100 can provide a variety of benefits, such as providing guidance to assist a physician in performing a procedure (such as fluid management status or conditions, instrument tracking, instrument alignment information, etc.), enabling a physician to perform a procedure from an ergonomic position without the need for awkward arm motions and/or positions, enabling a single physician to perform a procedure with one or more medical instruments, avoiding radiation exposure (such as associated with fluoroscopy techniques), enabling a procedure to be performed in a single operative setting, or providing continuous suction to remove an object more efficiently (such as to remove a kidney stone), among other examples. For example, the medical system 100 can provide guidance information to assist a physician in using various medical instruments to access a target anatomical feature while minimizing bleeding and/or damage to anatomy (such as critical organs or blood vessels).

Further, the medical system 100 can provide non-radiation-based navigational and/or localization techniques to reduce physician and patient exposure to radiation and/or reduce the amount of equipment in the operating room. Moreover, the medical system 100 can provide functionality that is distributed between two or more of the control system 50, the fluid management system 30, and the robotic system 10, which may be independently movable. Such distribution of functionality and/or mobility can enable the control system 50, the fluid management system 30, and/or the robotic system 10 to be placed at locations that are optimal for a particular medical procedure, which can maximize working area around the patient 7 and/or provide an optimized location for the physician 5 to perform a procedure.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the various communication interfaces of the systems of FIG. 2 can be configured to communicate with one or more device, sensors, or systems, such as over a wireless and/or wired network connection. In some implementations, the various communication interfaces can implement a wireless technology such as Bluetooth, Wi-Fi, near field communication (NFC), or the like. Furthermore, in some implementations, the various components of the medical system 100 can be connected for data communication, fluid exchange, power exchange, and so on via one or more support cables, tubes, or the like.

The control system 50, fluid management system 30, and/or robotic system 10 can include certain user controls 255 (such as a controller 55), which may comprise any type of user input (and/or output) devices or device interfaces, such as one or more buttons, keys, joysticks, handheld controllers (such as video-game-type controllers), computer mice, trackpads, trackballs, control pads, and/or sensors (such as motion sensors or cameras) that capture hand gestures and finger gestures, touchscreens, and/or interfaces or connectors therefore. Such user controls are communicatively and/or physically coupled to respective control circuitry.

In some implementations, a user can manually manipulate a robotic arm 12 of the robotic system 10 without using electronic user controls. For example, during setup in a surgical operating room, a user may move the robotic arms 12 and/or any other medical instruments to provide desired access to a patient. The robotic system 10 may rely on force feedback and inertia control from the user to determine appropriate configuration of the robotic arms 12 and associated instrumentation.

The fluid management system 30 may include one or more pumps 237, vacuums 230, flow meters, valve controls, and/or other fluid- or flow-control components (such as sensor devices, such as pressure sensors) in order to provide controlled irrigation and/or aspiration or suction capabilities for a medical instrument. To facilitate the fluid management system 30, the fluid management system 30 can include various components or subsystems. For example, the fluid management system 30 can include the control electronics or circuitry 231, as well as one or more I/O components 238, power supplies or supply interfaces 239, and/or communication interfaces 234. In some implementations, irrigation and aspiration capabilities can be delivered directly to or from a medical instrument through separate cable(s). In some implementations, the control circuitry 251 of the control system 50 (or the control circuitry 231 fluid management system 30 or the control circuitry 211 of the robotic system to 10) can generate and provide one or more signals to the fluid management system 30 to indicate how high (or low) the irrigation pressure level from an irrigation fluid source 332 can go, wherein such signals can be translated by the fluid management system 30 into irrigation outputs with respect to operation of the pump(s) 237 and/or other flow-control device(s) associated with the irrigation fluid source 332.

Any of the control circuitry 231, the control circuitry 251, and the control circuitry 211 may be configured to control the pump(s) 237 and/or the vacuum 230 to provide irrigation pressure limitation in accordance with aspects of the present disclosure. Any of the medical instruments 32, 40, 48 shown in the system 100 may be attached to the pump(s) 237, which may facilitate irrigation fluid flow. Although FIG. 2 includes the pump(s) 237, in some implementations, irrigation fluid flow is achieved without the use of pumps, wherein such flow is driven primarily by gravitational potential force. The pump(s) 237 is attached to the irrigation source 332, which provides irrigation fluid (such as a saline solution) to be pumped through one or more of the medical instruments 32, 40, 48 and into the treatment site. In some examples, the pump(s) 237 may be a peristaltic pump(s). In some implementations, the pump(s) 237 can be replaced with a vacuum that is configured to apply a vacuum pressure to draw the irrigation fluid from the irrigation fluid source 332 and out through the respective coupled medical instrument.

One or more of the percutaneous-access and/or direct-entry instruments implemented in connection with the systems 100, 200 may be fluidly coupled or connected to a vacuum 230 configured to facilitate fluid aspiration. For example, the vacuum 230 can be configured to apply a negative pressure to draw fluid out of the treatment site. The vacuum 230 may be connected to a collection container into which withdrawn fluid is collected. In some implementations, aspiration suction may be facilitated by one or more pumps rather than a vacuum. In some other implementations, aspiration may be primarily passive, rather than through active suction. Therefore, it should be understood that implementations of the present disclosure may not include vacuum components.

In some aspects, directed fluidics can be applied to a medical procedure so that irrigation (inflow) enters the treatment site through a first channel of a first medical instrument (such as a percutaneous-access instrument) and aspiration (outflow) exits the treatment site through a second channel of the first medical instrument. In some implementations, irrigation and aspiration can both be active. In some implementations, irrigation and aspiration can be managed to produce desirable flow characteristics. In some implementations, a second medical instrument that does not provide irrigation or aspiration can also be used during the procedure, for example, to break up the object being removed.

In some other aspects, directed fluidics can be applied to a medical procedure so that irrigation (inflow) enters the treatment site through a first medical instrument (such as an endoscope) while aspiration (outflow) exits the treatment site through a second medical instrument (such as a catheter). This can create a controlled flow from the first instrument towards the second instrument. The controlled flow can facilitate object removal. The first medical instrument can be inserted into the treatment site antegrade of the object to be removed, while the second medical instrument can be inserted into the treatment site retrograde of the object. In some implementations, the first medical instrument may be inserted into the treatment site (such as the kidney) through a patient lumen (such as the ureter) and the second medical instrument may be inserted into the treatment site percutaneously, or vice-versa.

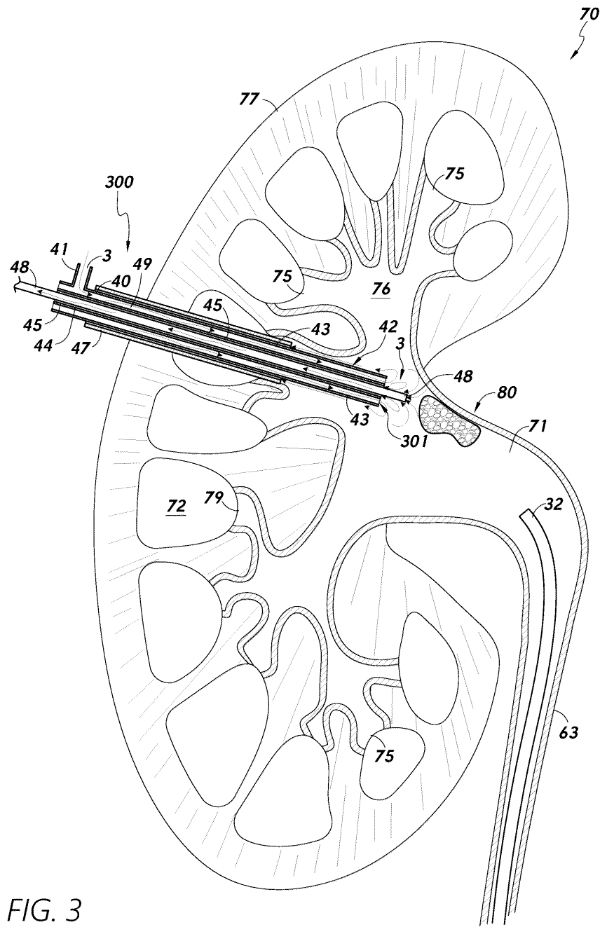

FIG. 3 shows an example medical procedure using directed fluidics, according to some implementations. For example, a device or system similar to the percutaneous-access device or assembly 40 may be used to provide irrigation (inflow) to a treatment site, such as an internal calyx network of a kidney 70. Fluid irrigation and aspiration, which may be referred to as “fluidics” herein, can represent an important component of certain medical procedures, such as percutaneous nephrolithotomy (PCNL). For example, during PCNL, fluidics may be applied to clear stone dust, small fragments, and thrombus from the treatment site as well as the visual field provided by the medical instrument(s). For example, with respect to the implementation of FIG. 3, irrigation fluid 3 can be provided through a channel 49 of the percutaneous-access instrument 40. Aspiration (outflow) may exit the treatment site through one or more passive and/or active outflow channels, which may or may not be associated with the percutaneous-access instrument 40. In some implementations, irrigation and aspiration can both be active.

In some aspects, the medical procedure depicted by FIG. 3 may be a PCNL procedure. The illustrated renal anatomy includes an object disposed in the calyx network of the kidney 70, wherein the object 80 can be any object that is targeted for removal, such as a kidney stone. In the illustrated example, the medical instrument assembly 300 includes a percutaneous-access laparoscope or nephroscope 40. Although described as a nephroscope below for convenience, it should be understood that the device 40 may be any type of medical instrument. The nephroscope 40 can be inserted percutaneously into the kidney 70 through an access sheath 47. According to some implementations, the access sheath 47 may be placed by first accessing the treatment site with a rigid needle and using a dilator to dilate the percutaneous-access path and place the sheath 47. The nephroscope 40 can include a working channel 44 within an inner shaft or wall 45, though which various tools can be inserted, such as a catheter 48. In some implementations, a lithotripter (such as an ultrasonic lithotripter) may be inserted through the working channel 44 of the nephroscope 40. The nephroscope 40 can also include an optic device (not shown) configured to allow a surgeon to visualize the treatment site.

The catheter 48 may be navigated within the kidney 70 by torquing the catheter 48 and/or nephroscope 40 towards the object 80. In some implementations, the object or stone 80 may be broken-up using a lithotripter (not shown) and removed in smaller fragments through the percutaneous-access catheter 48. The lithotripter may be advanced to the treatment site through percutaneous or direct entry (such as through the nephroscope 40 or through a sheath through which the scope 32 is advanced).

As illustrated with arrows in FIG. 3, irrigation (such as a saline solution) can be applied to the treatment site (such as the kidney 70) through the percutaneous-access instrument 40. The irrigation fluid 3 may enter the nephroscope 40 through an irrigation port 41 and exit through a distal end 301 into the kidney 70. Irrigation can be used to clear stone dust and small fragments from the field of view of, for example, the scope 32 or other image or viewing device to allow the surgeon to visualize the treatment site, as well as to distend the kidney 70 to allow access to the object 80. In the illustrated example, aspiration is also applied to the treatment site through the medical instrument 40. As shown, fluid can be removed from the kidney 70 through the access sheath 47 (such as between the outer shaft 43 of the nephroscope and the sheath 47) and/or through the working channel 44 of the nephroscope (such as between the inner shaft 45 of the nephroscope and the catheter 48). In some implementations, aspiration may be provided through a channel in a lithotripter. In some instances, aspiration is pulled (actively) through one or more outflow channels and/or permitted to passively flow through one or more outflow channels. For example, active aspiration or suction may be drawn through the catheter 48. In some implementations, fluidics are applied during substantially the entire procedure.

The fluidics applied during the procedure can establish a fluid flow as illustrated by the arrows in FIG. 3. Initially, fluid can flow outward from the distal tip 301 of the nephroscope 40 towards the object 80. Aspiration through the access sheath 47 and/or working channel 44 can cause fluid flow back towards the nephroscope or sheath. As illustrated, in the region of the object 80, the flow may be both directed toward and away from the object 80 with respect to the distal end 301 of the nephroscope 40. Where the available fluid outflow channels are insufficient to remove a flow of fluid equal to the irrigation flow into the treatment site, risks of over-filling the kidney can be present.

During a ureteroscopic lithotripsy procedure, the ureteroscope 32 may enter the kidney 70 through the ureter 63 and use stone-retrieval basket(s) and/or lithotripter(s) to relocate and break down kidney stones, respectively. For example, a lithotripter can be deployed through a working channel of the ureteroscope 32 and used to break the stone 80 into fragments, which may be aspirated through the catheter 48.

Irrigation and/or aspiration can be managed to produce desirable flow characteristics resulting in desirable distension conditions for the target organ or anatomy. In some implementations, irrigation (inflow) enters the treatment site through a first medical device (such as the percutaneous-access instrument 40 and/or catheter 48 disposed therein). In some implementations, the percutaneous-access instrument 40 and/or catheter 48 can be inserted into the treatment site antegrade of an object (such as a kidney stone) 80 to be removed, whereas another medical instrument (such as an endoscope) 32 can be inserted into the treatment site retrograde of the object 80. Although FIG. 3 shows the irrigation fluid 3 as provided to the treatment site percutaneously, in some other implementations, irrigation may be provided via a medical instrument that accesses the treatment site through direct entry (such as the ureteroscope 32). Any of the percutaneous-access instruments 40, 48 or direct-entry instruments 32 may be robotically controlled as described above with reference to FIGS. 1 and 2. Accordingly, aspects of the present disclosure can be employed robotically in some implementations.

In some implementations, the percutaneous-access instrument 40 may provide irrigation with a sufficiently high inflow rate without causing turbulence. This may allow the treatment site (such as the kidney 70) to fill up with fluid without displacing the object 80. In some other implementations, the irrigation and/or aspiration rate(s) can be modulated to improve stone displacement or stabilization or to intentionally create turbulence so that the irrigation reaches all corners of the treatment site. For example, a gentle alternating cycle of irrigation and aspiration can create a lavage-like effect to preferentially pull large stone debris away from calyces and towards the aspiration site(s). Alternatively, short pulsatile inflow and outflow could be used to create turbulence and ensure that smaller and lighter stone fragments do not settle on the floor of the treatment site, but instead remain floating in the irrigation fluid and eventually are aspirated with the outflow.

The percutaneous-access catheter 48 can be an articulable catheter that is introduced via percutaneous access into the treatment site (such as the calyx network of the kidney 70). The catheter 48 can be navigated within the kidney 70. For example, the catheter 48 may be configured to be inserted and retracted into the treatment site and/or to articulate (such as to bend) therein. In some implementations, the catheter 48 can include pull-wires for controlling articulation. For example, four pull-wires may be oriented in the four orthogonal directions to enable articulation of the catheter 48. Other methods for permitting articulation of the catheter are also possible. The catheter 48 can include, for example, an aspiration lumen (or channel). The aspiration lumen can be fluidly coupled to a pump or vacuum device (such as an external pump). The pump or vacuum may generate negative pressure that causes flow from the treatment site into the catheter. The aspiration function may be able to be toggled (such as on and off) and adjusted by the user or system. In some implementations, the aspiration lumen may be used for irrigation as well.

The catheter 48 can provide various functions during an object removal procedure, such as stone stabilization during lithotripsy. For example, if the stone 80 is larger than the aspiration lumen of the catheter, the stone can be held at the distal face of the aspiration lumen, thus stabilizing the stone while it is broken down to dust and smaller fragments. Active aspiration may hold the stone to the distal face of the catheter 48. In some cases, a stone being extracted can substantially seal off the catheter 48, thereby causing the stone to be held by the catheter due to the pressure differential. This may provide the user with a less-mobile target for lithotripsy. Moreover, the catheter 48 can improve visibility of the treatment site by removing stone dust from the kidney. This can provide the user with improved visibility (such as continuously adequate visibility), for example, from an imaging device inserted into the treatment site (such as a camera associated with the scope 32).

The catheter 48 can remove stone dust and fragments, wherein the fluid flow carries fluid and debris into the catheter 48 for removal therethrough. Generally, the debris may be cleared as it is generated (such as while the stone is being broken up). The removal of debris via the catheter 48 can take the place of the removal of fragments via ureteroscopic basketing, which can be relatively time consuming due to the difficulty of closing the basket around the stone, and due to the need to remove and re-insert the ureteroscope during each fragment removal. Therefore, using the catheter for stone removal can result in a more efficient removal procedure. Removing stone debris via the catheter can also reduce the risk of the stone fragment(s) injuring tissue compared to certain alternative stone removal methods, such as removal of stones through the ureter.

The catheter 48 can be used in several ways during a procedure. For example, the catheter 48 can be mobile throughout the procedure. The catheter can navigate around the treatment site to target specific stones or fragments in order to constrain them during lithotripsy, while also aspirating dust or debris. As another example, the catheter 48 can be initially stationary during the procedure and the scope 32 can be used to relocate stones to the catheter 48. The stones may be broken down at the catheter 48. At a later time during the procedure, the catheter 48 may be navigated through the treatment site to pick up remaining debris. As another example, the catheter 48 may be inserted (such as percutaneously) only when required, for example, during procedure escalation.

In some other aspects, directed fluidics may include the separation of the point(s) of inflow (irrigation) from the point(s) of outflow (aspiration). For example, the inflow can be directed towards the point of outflow by deflecting the distal end of a first medical instrument (such as the endoscope 32) towards a second medical instrument (such as the percutaneous-access instrument 40) such that the fluid flow is towards the second medical instrument. This may be accomplished robotically and/or manually with the systems and instruments described above with reference to FIGS. 1 and 2. In some implementations, the point of outflow (aspiration) may be a single or concentrated point. More specifically, the point of outflow may be configured to provide high flow with high velocities so as to cause fragments to be pulled towards the point of outflow.

As described with reference to FIGS. 1-3, fluidics can be used for various purposes such as, for example, to achieve anatomical distension (such as for endoscopic vision), maintain suitable intrarenal pressures (such as to prevent damage to the anatomy), or move around objects (such as urinary stones) within the anatomy. Improper management of irrigation and/or aspiration can adversely affect the health of the patient and/or efficacy of the medical procedure. For example, under-pressurization of an anatomy can result in insufficient distension that is otherwise needed to visualize and/or access certain parts of the anatomy using various medical instruments. Thus, some physicians (or technicians) may rely on fluidics simulations to predict the achievable levels of distension at various parts of an anatomy when planning a percutaneous access procedure.

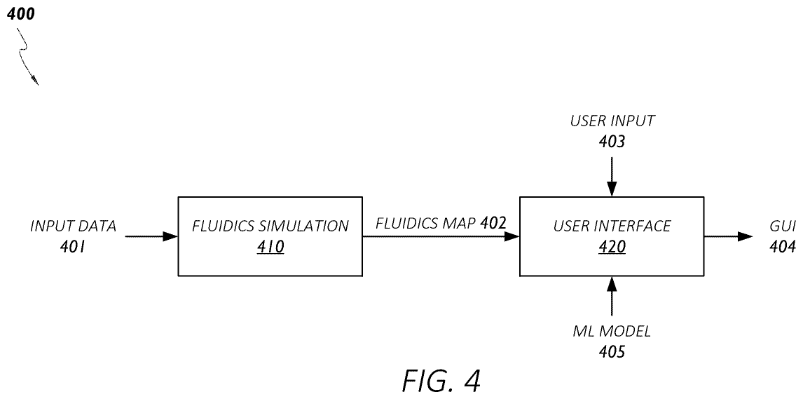

FIG. 4 shows a block diagram of an example controller 400 for a medical system, according to some implementations. In some implementations, the medical system may be one example of the medical system 100 of FIG. 1. With reference to FIG. 2, the controller 400 may be one example of any of the control circuitry 211, 231, and/or 251. The controller 400 is configured to generate a graphical user interface (GUI) 404 that allows a user of the medical system (such as a physician or a technician) to plan and/or perform a percutaneous access procedure. For example, the GUI 404 may be presented on the display 56 of FIGS. 1 and 2.

The controller 400 includes a fluidics simulation component 410 and a user interface component 420. The fluidics simulation component 410 is configured to generate a fluidics map 402 based on input data 401 representing a 3D mapping of an anatomy (such as the kidney 70 of FIGS. 1 and 3). For example, the input data 401 may be captured or acquired using one or more anatomical imaging technologies. Example suitable imaging technologies include CT, X-ray, fluoroscopy, PET, PET-CT, CT angiography, CBCT, 3DRA, SPECT, MRI, OCT, or ultrasound, among other examples. In some aspects, the input data 401 may include tomograms depicting a 3D image of the anatomy. In some implementations, the tomograms may be acquired during a preoperative phase of a medical procedure (such as via a CT imaging system). In some other implementations, the tomograms may be acquired during an intraoperative phase of a medical procedure (such as via a fluoroscopy or CBCT imaging system).

The fluidics simulation component 410 may extract a polygon mesh from the input data 401 and perform a fluidics simulation on the polygon mesh to produce the fluidics map 402. A polygon mesh is a collection of vertices (points in 3D space), triangles (line segments connecting adjacent vertices), and faces (flat surfaces bounded by edges) that defines the shape or boundaries of a polyhedral object (such as the kidney 70). The fluidics simulation component 410 may implement any known or existing fluidics simulation techniques or algorithms for generating the fluidics map 402. In some implementations, the fluidics map 402 may be a velocity map showing streamlines and/or velocities of predicted fluid flow at various portions of the anatomy. In some other implementations, the fluidics map 402 may be a pressure map showing predicted fluid pressures at various portions of the anatomy.

The user interface component 420 is configured to generate the GUI 404 based, at least in part, on the fluidics map 402. In some aspects, the GUI 404 may display the fluidics map 402 so that the user can make an informed decision about how to percutaneously access a target within the anatomy (such as the kidney stone 80 of FIGS. 1 and 3). In some implementations, the user may analyze the fluidics map 402 to determine the achievable levels of distension at various portions of the anatomy. For example, portions of an anatomy predicted to have higher fluid volume, velocities, and/or pressures can achieve greater distension compared to portions of the anatomy in which the fluid volume, velocities, and/or pressures are more restricted. Thus, the user may select a location on the anatomy for percutaneous entry that is predicted to achieve at least a threshold amount of distension suitable for visualization and/or movement of instruments within the anatomy. The percutaneous entry location represents a portion of the anatomy (such as a kidney pole) through which the user inserts an instrument percutaneously (such as the percutaneous-access instrument 40 and/or the catheter 48 of FIGS. 1 and 3).

In some implementations, the user interface component 420 may further receive user input 403 for controlling one or more instruments within the anatomy. With reference for example to FIG. 2, the user input 403 may be provided via the I/O components 258 and/or the controller 55. For example, the user may provide user input 403 to drive a scope (such as the scope 32 of FIGS. 1 and 3) to touch or tag the percutaneous entry location from inside the anatomy. To assist the user with inserting the percutaneous-access instrument into the anatomy through the tagged location, the user interface 420 may display a visualization on the GUI 404 to indicate an alignment of an orientation of the percutaneous-access instrument relative to a target trajectory (such as a desired access path), a visualization to indicate a progress of inserting the percutaneous-access instrument towards the target location, and/or other information. With the percutaneous-access instrument inserted into the anatomy, the user can use the instrument and/or another medical instrument to extract the target (such as described with reference to FIGS. 1-3).

In some aspects, the user interface component 420 may at least partially automate the planning of the percutaneous access procedure based on a machine learning (ML) model 405. For example, the ML model 405 may be trained using fluidics maps 402 and user inputs 403 collected by the controller 400 over a large volume of medical procedures. More specifically, the trained ML model 405 may infer a percutaneous access plan from the fluidics map 402 for the current percutaneous access procedure. In some implementations, the percutaneous access plan may include one or more recommended percutaneous access locations on the anatomy. In some other implementations, the percutaneous access plan may include recommended poses for one or more instruments within the anatomy (such as the scope 32, the percutaneous-access instrument 40, and/or the catheter 48). The user interface component 420 may display or otherwise present the percutaneous access plan on the GUI 404. For example, the recommended percutaneous access location and/or recommended instrument poses may be displayed as an overlay on the fluidics map 402 in the GUI 404.

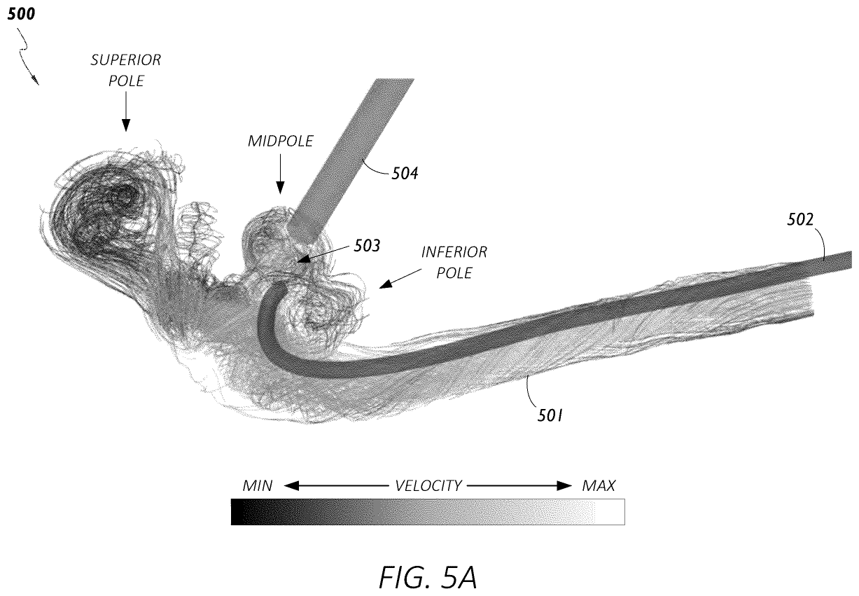

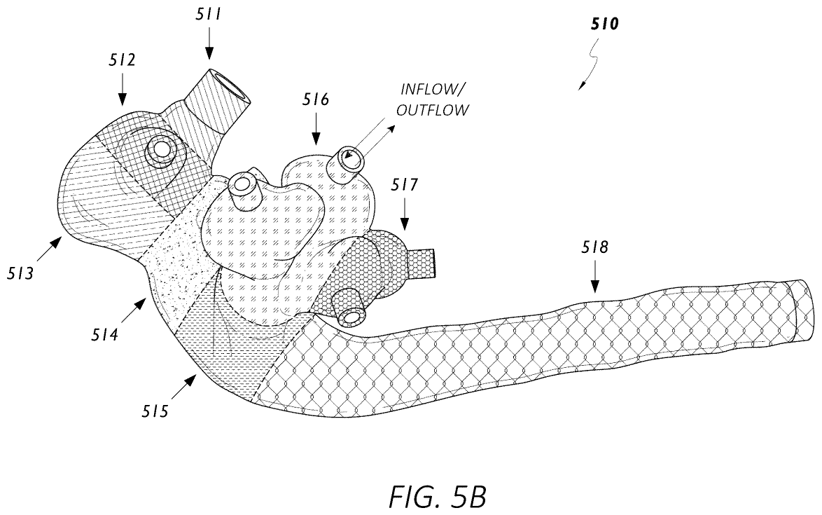

FIG. 5A shows a graphical interface 500 depicting an example percutaneous access plan, according to some implementations. More specifically, the percutaneous access plan includes a recommended pose of a direct-entry instrument 502 and a recommended pose of a percutaneous-access instrument 504 overlaid on a fluidics map 501 of a kidney. In some implementations, the graphical interface 500 may be generated by the controller 400 of FIG. 4. With reference to FIG. 4, the graphical interface 500 may be one example of the GUI 404 and the fluidics map 501 may be one example of the fluidics map 402. In the example of FIG. 5A, the fluidics map 501 is a velocity map depicting streamlines of fluid flow and their respective velocities in various regions of the kidney. More specifically, streamlines having lighter shades or colors represent higher velocity fluid flow whereas streamlines having darker shades or colors represent lower velocity fluid flow.

As shown in FIG. 5A, the kidney anatomy includes a superior (or “upper”) pole, an inferior (or “lower”) pole, and a midpole (between the super and inferior poles). Each kidney pole (also referred to as a “renal pole”) represents a different region of the kidney having distinct fluid flow characteristics. For example, the fluidics map 501 predicts the velocity of fluid flow to be higher in the midpole than any of the superior or inferior poles while maintaining safe levels of intrarenal pressure. Accordingly, the midpole of the kidney is likely to achieve the greatest amount of distension under fluid irrigation (compared to the superior and inferior poles) and is therefore selected by the controller 400 as the recommended percutaneous entry location. In the example of FIG. 5A, the percutaneous-access instrument 504 is overlaid on the midpole of the kidney to indicate the selection of the percutaneous entry location.

As described with reference to FIG. 4, the controller 400 (or the user interface component 420) may select the percutaneous entry location based on the ML model 405. More specifically, the ML model 405 may be trained on historical fluidics simulations and user input data to predict which of the kidney poles will have the greatest amount of distension based on the fluidics map 501 and/or to infer a positioning of the instruments 502 and 504 for percutaneously accessing a target within the kidney (such as a kidney stone) so that the percutaneous-access instrument 504 is inserted through the kidney pole predicted to have the greatest amount of distension. The instruments 502 and 504 are positioned around a location of a target 503 for treatment (also referred to as the “target treatment location”). In some implementations, the recommended poses of the instruments 502 and 504 may ensure optimal anatomical distension, visual clarity, and/or system performance (such as by configuring system presets that allow for the greatest range of articulation or arm angles of the instruments 502 and/or 504).

In some implementations, an anatomy may be segmented into different regions, for example, to obtain more granular information about the flow dynamics inside the anatomy. For example, FIG. 5B shows a segmentation map 510 depicting an example segmentation of the kidney shown in FIG. 5A. In the example of FIG. 5B, the kidney is segmented into 8 distinct regions 511-518 each shown with a different pattern or design. With reference for example to FIG. 5A, regions 511-513 overlap the superior pole, regions 514-516 overlap the midpole, region 517 overlaps the inferior pole, and region 518 overlaps the ureter. The segmentation map 510 also depicts several cylindrical tubes protruding from the regions 511, 512, 514, 516, and 517 to indicate potential percutaneous entry locations. For example, after comparing the flow characteristics of the different regions 511-518, the ML model 405 may select one of the cylindrical tubes as the recommended percutaneous entry location. As shown in FIG. 5B, the selected percutaneous entry location is indicated by the bidirectional arrows depicting fluid inflow and outflow (such as from the percutaneous-access instrument 504 of FIG. 5A).

Aspects of the present disclosure recognize that fluidics simulations may not be available for some medical procedures. For example, some medical procedures may rely on lower resolution anatomical imaging and/or mapping techniques, during an intraoperative phase of a medical procedure, in lieu of 3D images (such as CT scans) that are otherwise needed for fluidics simulations. In some implementations, a user may inject a contrast dye into an anatomy to capture a pyelogram of the anatomy. In some other implementations, a user may trace the anatomy using a direct-entry instrument (such as the scope 32) while mapping the position and/or movement of the instrument via one or more sensors used for navigating the instrument within the anatomy (also referred to as a sensor map). For example, the sensors may be disposed on the instrument and may output sensor data to a controller for a medical system (such as the controller 400 of FIG. 4). Example suitable sensor technologies that can be used for generating a sensor map include electromagnetic (EM) sensors for tracking a pose of the instrument and cameras for visualization within the anatomy, among other examples.

Aspects of the present disclosure further recognize that various anatomies can be parameterized and separated into classes based on known anatomical features which can be correlated or otherwise associated with fluidics simulations performed on such anatomies. For example, various regions of a kidney can be classified as “superior poles,” “inferior poles,” or “midpoles” based on their relative locations and/or structure (such as shown in FIG. 5A), and kidney poles can be further segmented into more granular regions (such as shown in FIG. 5B). Thus, a classification of kidney poles is an example feature that can be extracted from anatomical maps of various resolutions, including CT scans, pyelograms, and sensor maps, among other examples. Further, the shapes, sizes, and/or presence of various anatomical features can be used to classify anatomies into various “types.” For example, the presence (or lack) of a midpole and/or the size or shape of a renal pelvis (or infundibulum) can be used to classify a kidney into one of several kidney types.

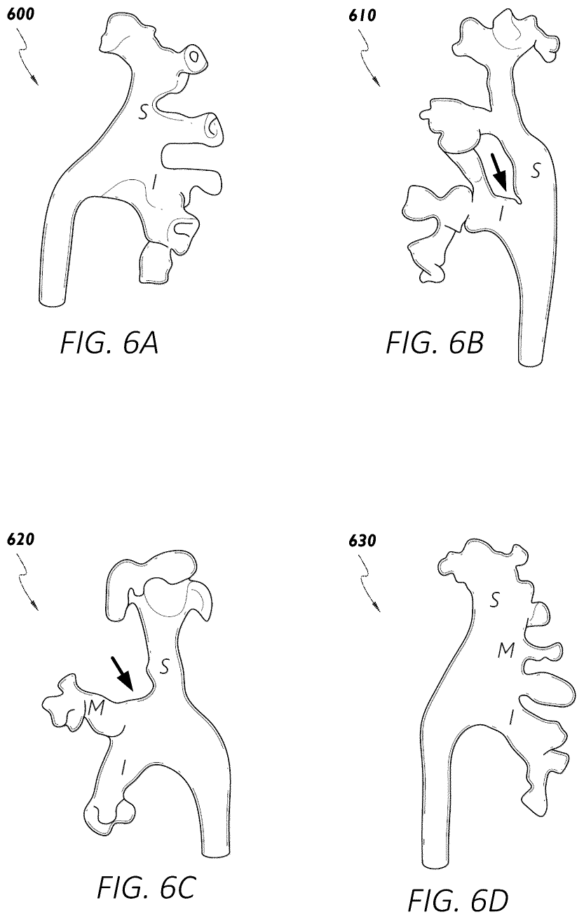

FIGS. 6A-6D show various example kidney types 600-630, respectively, according to some implementations. As shown in FIGS. 6A-6D, each of the kidney types 600 and 610 has a superior pole (labeled “S”) and an inferior pole (labeled “I”), whereas each of the kidney types 620 and 630 has a clearly defined midpole (labeled “M”) in addition to a superior pole and an inferior pole. Further, each of the kidney types 600 and 630 has a single renal pelvis (which is the region of the kidney connecting the calyces to the ureter), whereas each of the kidney types 610 and 620 has a divided renal pelvis (where the bifurcation of the renal pelvis is highlighted by an arrow). Thus, any kidney can be classified as one of the kidney types 600-630 based on whether the kidney has (or lacks) a midpole and whether the kidney has a divided renal pelvis (or a single renal pelvis).

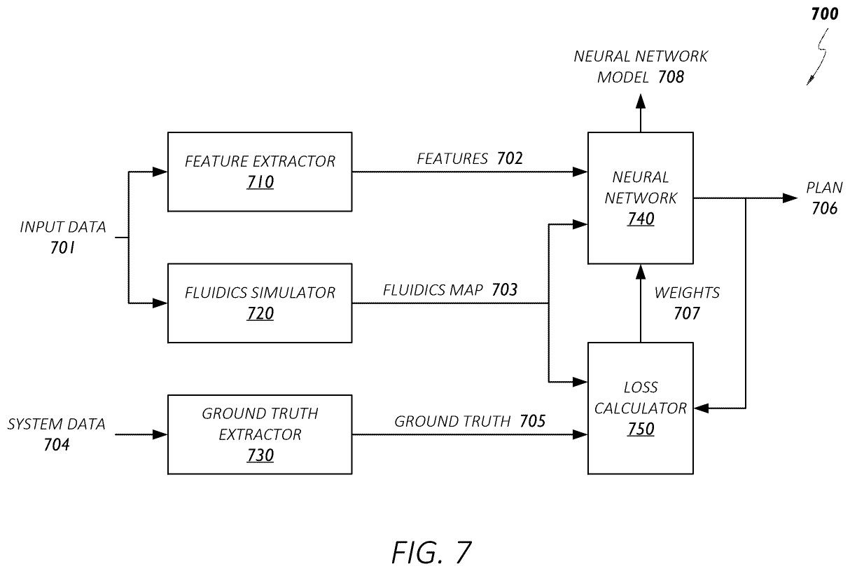

FIG. 7 shows a block diagram of an example machine learning system 700, according to some implementations. The machine learning system 700 is configured to produce a neural network model 708 based, at least in part, on input data 701 representing a large volume of anatomical maps of various resolutions. For example, the input data 701 may be received via the controller 400 of FIG. 4 and/or any of the medical systems 100 or 200 of FIGS. 1 and 2, respectively. In some implementations, the neural network model 708 may be one example of the neural network model 405 of FIG. 4. More specifically, the neural network model 708 may be trained to infer a percutaneous access plan from new input data representing an anatomical map (such as the input data 401 of FIG. 4).

The machine learning system 700 includes a feature extractor 710, a fluidics simulator 720, a ground truth extractor 730, a neural network 740, and a loss calculator 750. The feature extractor 710 is configured to extract one or more features 702 from the input data 701. In some implementations, the input data 701 may represent a high-fidelity anatomical map. Example suitable high-fidelity maps include 3D images (such as CT scans) of the anatomy. In some other implementations, the input data 701 may represent a medium-fidelity anatomical map. Example suitable medium-fidelity maps include 2D images (such as pyelograms) of the anatomy. Still further, in some implementations, the input data 701 may represent a low-fidelity anatomical map. Example suitable low-fidelity maps include sensor maps of the anatomy.

In some implementations, the feature extractor 710 may determine the features 702 through analysis of the input data 701 using one or more image processing techniques. Example suitable image processing techniques include segmentation, machine learning, and statistical analysis, among other examples. As used herein, the term “segmentation” refers to various techniques for partitioning a digital image into groups of pixels or voxels (also referred to as “image segments”) based on related characteristics or identifying features. The number and/or types of features 702 that can be extracted by the feature extractor 710 may depend on the resolution or fidelity of the input data 701. Generally, more features can be extracted from higher fidelity maps than lower fidelity maps. Further, the features extracted from higher fidelity maps may be more precise and/or accurate than the features extracted from lower fidelity maps.

Example features 702 that can be extracted from low-, medium-, and high-fidelity anatomical maps (such as sensor maps, pyclograms, and CT scans, among other examples) include a classification of anatomical regions (such as the kidney poles described with reference to FIGS. 5A and 5B), an anatomy type (such as the kidney types 600-630 described with reference to FIGS. 6A-6D), a position of a target (such as a kidney stone) within the anatomy, one or more dimensions (or a size) of the target, and a width (or diameter) of a direct-entry instrument within the anatomy (such as the scope 32 of FIGS. 1 and 3). Example features 702 that can be additionally extracted from medium- and high-fidelity anatomical maps (such as CT scans and pyelograms) include a volume of the anatomy, a diameter of a lumen of the anatomy (such as a ureter), dimensions of one or more regions of the anatomy (such as a renal pelvis or infundibulum), and a pose of the direct-entry instrument. Example features 702 that can be additionally extracted from high-fidelity anatomical maps (such as CT scans) include a polygon mesh of the anatomy and a polygon mesh of the target.

In some implementations, the polygon meshes of the anatomy and/or the target may be extracted by the fluidics simulator 720. The fluidics simulator 720 is configured to generate a fluidics map 703 based on the input data 701 (such as where the input data 701 represents a high-fidelity anatomical map). In some implementations, the fluidics simulator 720 and the fluidics map 703 may be examples of the fluidics simulation component 410 and the fluidics map 402, respectively, of FIG. 4. For example, the fluidics simulator 720 may extract the polygon meshes of the anatomy and the target and perform a fluidics simulation on the polygon meshes to produce the fluidics map 703. In some implementations, the fluidics map 703 may be a velocity map showing streamlines and/or velocities of predicted fluid flow at various portions of the anatomy. In some other implementations, the fluidics map 703 may be a pressure map showing predicted fluid pressures at various portions of the anatomy.

The ground truth extractor 730 is configured to extract ground truth data 705 from system data 704 associated with the input data 701. The system data 704 may include any runtime data that a medical system generates or collects during a medical procedure. Example suitable runtime data includes data representing the poses of the instruments of the medical system, poses of the robotic arms or manipulators of the medical system, user interface (UI) commands, system status, video data, and audio data, among other examples. The ground truth data 706 describes how a user obtained percutaneous access to the target within the anatomy having the extracted features 702. In some implementations, the ground truth data 706 may include a percutaneous entry location on the anatomy (such as the midpoles shown in FIGS. 5A and 5B). In some other implementations, the ground truth data 706 may include a position of the target within the anatomy (such as the target treatment location 503 of FIG. 5A) and/or poses of one or more instruments used in treating the target (such as the poses of the instruments 502 and 504 of FIG. 5A). Still further, in some implementations, the ground truth data 706 may include a fluidics map of the anatomy.

Aspects of the present disclosure recognize that a percutaneous access procedure can be subdivided into 3 phases: a target selection phase (where a target location within the anatomy is selected or designated for percutaneous access), a site selection phase (where a needle is placed on the surface of the patient's skin and aligned with the target location), and a needle insertion phase (where the needle is inserted percutaneously to rendezvous with the target location). For robotic-assisted procedures, the medical system may provide the user with one or more interfaces (such as the GUI 404 of FIG. 4) for selecting a target location during the target selection phase, selecting a needle insertion site during the site selection phase, and controlling or manipulating a robotic system (such as the robotic system 10 of FIGS. 1 and 2). In some implementations, the system data 704 may include the selected target location, the selected needle insertion site, and/or a robot workspace (such as a range of robotic arm movements) recorded by the medical system during a percutaneous access procedure.

In some aspects, the machine learning system 700 may train the neural network 740 to reproduce the ground truth data 705 based on the extracted features 702 and the fluidics map 703. Deep learning is a particular form of machine learning in which the inferencing and training phases are performed over multiple layers. Deep learning architectures are often referred to as “artificial neural networks” due to the manner in which information is processed (similar to a biological nervous system). For example, each layer of an artificial neural network may be composed of one or more “neurons.” Each layer of neurons may perform a different transformation on the output data from a preceding layer so that the final output of the neural network results in the desired inferences. The set of transformations associated with the various layers of the network is referred to as a “neural network model.” Example suitable neural networks include convolutional neural networks (CNNs), recurrent neural networks (RNN), and long short-term memory (LSTM) networks, among other examples.

The neural network 740 receives the extracted features 702 and the fluidics map 703 and attempts to recreate the ground truth data 705. For example, the neural network 740 may form a network of connections across multiple layers of artificial neurons that begin with the features 702 and fluidics map 703 and lead to a percutaneous access plan 706 at its output. The connections are weighted to result in an output percutaneous access plan 706 that closely resembles the ground truth data 705 (and fluidics map 703). The training operation is performed over multiple iterations. In each iteration, the neural network 740 produces a percutaneous access plan 706 based on weighted connections across the layers of artificial neurons, and the loss calculator 750 updates the weights 707 associated with the connections based on an amount of loss (or error) between the percutaneous access plan 706 and the ground truth data 705 (and fluidics map 703). The neural network 740 may output the weighted connections as the neural network model 708 when certain convergence criteria are met (such as when the loss falls below a threshold level or a predetermined number of training iterations have been performed).

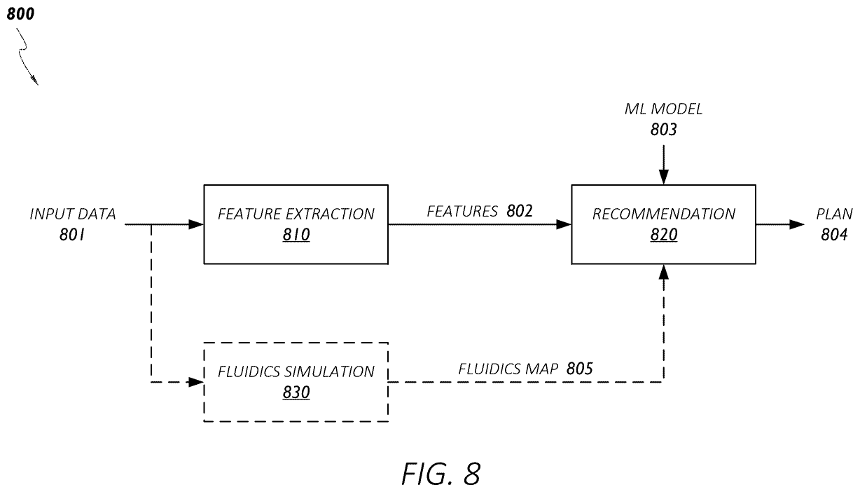

FIG. 8 shows a block diagram of an example recommendation system 800 for percutaneous access procedures, according to some implementations. In some implementations, the recommendation system 800 may be one example of the controller 400 of FIG. 4. More specifically, the recommendation system 800 is configured to generate a percutaneous access plan 804 for percutaneously accessing a target within an anatomy based on input data 801 representing a mapping of the anatomy.

The recommendation system 800 includes a feature extraction component 810 and a recommendation component 820. The feature extraction component 810 is configured to extract one or more features 802 from the input data 801. In some implementations, the feature extraction component 810 may be one example of the feature extractor 710 of FIG. 7. For example, the feature extraction component 810 may determine the features 802 through analysis of the input data 801 using one or more image processing techniques (such as segmentation, machine learning, and statistical analysis, among other examples). As described with reference to FIG. 7, the number and/or types of features 802 that can be extracted by the feature extraction component 810 may depend on the resolution or fidelity of the input data 801. For example, more features can be extracted from higher fidelity maps than lower fidelity maps, and the features extracted from higher fidelity maps may be more precise and/or accurate than the features extracted from lower fidelity maps.

In some implementations, the input data 801 may represent a low-fidelity anatomical map (such as a sensor map of the anatomy). In such implementations, the extracted features 802 may include a classification of anatomical regions (such as a classification of kidney poles), an anatomy type (such as a kidney type), a position of a target (such as a kidney stone) within the anatomy, one or more dimensions (or a size) of the target, and a width (or diameter) of a direct-entry instrument within the anatomy (such as the scope 32 of FIGS. 1 and 3). In some other implementations, the input data 801 may represent a medium-fidelity anatomical map (such as a pyelogram or other 2D image of the anatomy). In such implementations, the extracted features 802 may further include, in addition to the features that can be extracted from low-fidelity maps, a volume of the anatomy, a diameter of a lumen of the anatomy (such as a ureter), dimensions of one or more regions of the anatomy (such as a renal pelvis or infundibulum), and a pose of the direct-entry instrument. Still further, in some implementations, the input data 801 may represent a high-fidelity anatomical map (such as a CT scan or other 3D image of the anatomy). In such implementations, the extracted features 802 may further include, in addition to the features that can be extracted from medium-fidelity maps, a polygon mesh of the anatomy and a polygon mesh of the target.

In some aspects, the recommendation system 800 may further include a fluidics simulation component 830 to generate a fluidics map 805 based on input data 801 representing a high-fidelity anatomical map. In some implementations, the fluidics simulation component 830 may be one example of the fluidics simulation component 410 or the fluidics simulator 720 of FIGS. 4 and 7, respectively. For example, the fluidics simulation component 830 may extract polygon meshes of the anatomy and the target and perform a fluidics simulation on the polygon meshes to produce the fluidics map 805. In some implementations, the fluidics map 805 may be a velocity map showing streamlines and/or velocities of predicted fluid flow at various portions of the anatomy (such as shown in FIG. 5A). In some other implementations, the fluidics map 805 may be a pressure map showing predicted fluid pressures at various portions of the anatomy.

The recommendation component 820 is configured to infer the percutaneous access plan 804 from the extracted features 802 and/or the fluidics map 805 based on an ML model 803. In some implementations, the ML model 803 may be one example of the ML model 405 or the neural network model 708 of FIGS. 4 and 7, respectively. More specifically, the ML model 803 may be trained on fluidics simulations that predict anatomical distension in response to irrigation (such as described with reference to FIG. 7). In some implementations, the percutaneous access plan 804 may include a recommended percutaneous entry location on the anatomy (such as the midpoles shown in FIGS. 5A and 5B). In some other implementations, the percutaneous access plan 804 may include a position of the target within the anatomy (such as the target treatment location 503 of FIG. 5A) and/or recommended poses for one or more instruments to be used in treating the target (such as the poses of the instruments 502 and 504 of FIG. 5A). Still further, in some implementations, the percutaneous access plan 804 may include a fluidics map of the anatomy. In some aspects, the percutaneous access plan 804 may be displayed or presented on a graphical interface (such as the graphical interface 500 of FIG. 5).

Aspects of the present disclosure recognize that the recommendations provided in the percutaneous access plan 804 may depend on the amount and/or types of features 802 extracted from the input data 801. For example, in some implementations where the input data 801 represents a high- or medium-fidelity anatomical map, the recommendation component 820 may generate a percutaneous access plan 804 that includes a recommended percutaneous entry location, recommended poses for one or more instruments to be used in treating the target (or a position of the target within the anatomy), and/or a fluidics map of the anatomy. However, the ML model 803 may not be able to infer a fluidics map from the features extracted from low-fidelity anatomical map (such as sensor maps). In such implementations, the recommendation component 820 may generate a percutaneous access plan 804 that includes a recommended percutaneous entry location and/or recommended poses of one or more instruments to be used in treating the target (or a position of the target within the anatomy).

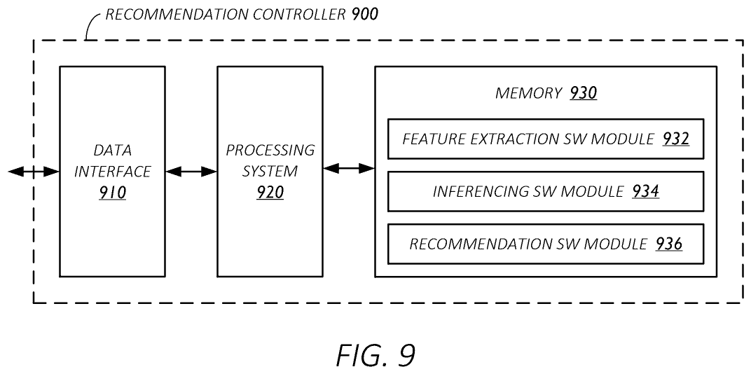

FIG. 9 shows another block diagram of an example controller 900 for a medical system, according to some implementations. In some implementations, the controller 900 may be one example of the controller 400 or the recommendation system 800 of FIGS. 4 and 8, respectively. More specifically, the controller 900 is configured to generate a percutaneous access plan for percutaneously accessing a target within an anatomy based on input data representing a mapping of the anatomy.

The controller 900 includes a data interface 910, a processing system 920, and a memory 930. The data interface 910 is configured to receive the input data representing a mapping of the anatomy. In some implementations, the input data may include a 3D image of the anatomy (such as a CT scan). In some other implementations, the input data may include a 2D image of the anatomy (such as a pyelogram). Still further, in some implementations, the input data may include sensor data received from one or more sensors disposed on an instrument within the anatomy (such as a direct-entry instrument).

The memory 930 may include a non-transitory computer-readable medium (including one or more nonvolatile memory elements, such as EPROM, EEPROM, Flash memory, or a hard drive, among other examples) that may store the following software (SW) modules: a feature extraction SW module 932 to extract, from the input data, a plurality of features including a position of a target within the anatomy; an inferencing SW module 934 to infer, from the plurality of features, a location on the anatomy for percutaneous entry based on a machine learning model trained on fluidics simulations that predict anatomical distension in response to irrigation; and a recommendation SW module 936 to generate a plan for percutaneously accessing the target via the location inferred from the plurality of features. Each of the software modules 932-936 includes instructions that, when executed by the processing system 920, causes the controller 900 to perform the corresponding functions.

The processing system 920 may include any suitable one or more processors capable of executing scripts or instructions of one or more software programs stored in the controller 900 (such as in the memory 930). For example, the processing system 920 may execute the feature extraction SW module 932 to extract, from the input data, a plurality of features including a position of a target within the anatomy. The processing system 920 may further execute the inferencing SW module 934 to infer, from the plurality of features, a location on the anatomy for percutaneous entry based on a machine learning model trained on fluidics simulations that predict anatomical distension in response to irrigation. The processing system 920 also may execute the recommendation SW module 936 to generate a plan for percutaneously accessing the target via the location inferred from the plurality of features.

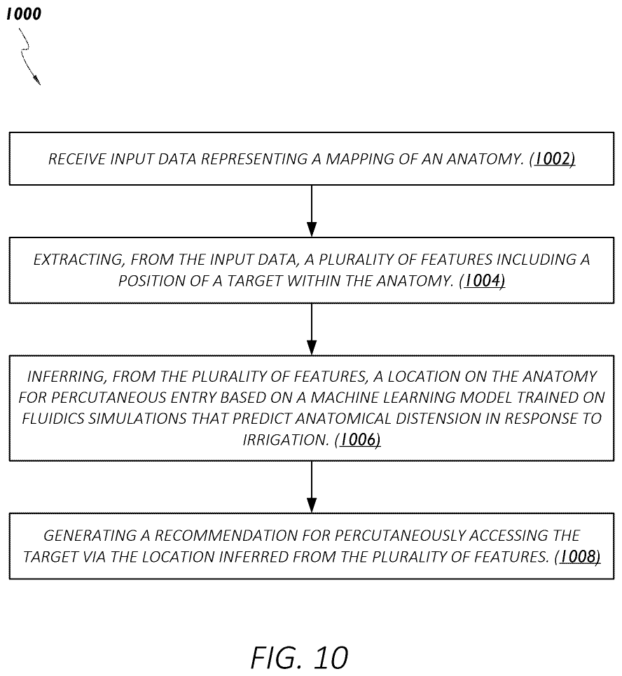

FIG. 10 shows an illustrative flowchart depicting an example operation 1000 for generating a percutaneous access recommendation, according to some implementations. In some implementations, the example operation 1000 may be performed by a controller for a medical system such as any of the controllers 400 or 900 of FIGS. 4 and 9, respectively, or the recommendation system 800 of FIG. 8.

The controller may receive input data representing a mapping of an anatomy (1002). In some implementations, the input data may include a 3D image of the anatomy. In some other implementations, the input data may include a 2D image of the anatomy. The controller may extract, from the input data, a plurality of features including a position of a target within the anatomy (1004). The controller may infer, from the plurality of features, a location on the anatomy for percutaneous entry based on a machine learning model trained on fluidics simulations that predict anatomical distension in response to irrigation (1006). The controller may further generate a plan for percutaneously accessing the target via the location inferred from the plurality of features (1008).

In some aspects, the plan may include a recommended pose of a first instrument configured for direct entry and a recommended pose of a second instrument configured for percutaneous access. In some implementations, the recommended poses of the first and second instruments may be inferred from the plurality of features based on the machine learning model. In some implementations, the input data may include sensor data received from one or more sensors disposed on the first instrument. In some implementations, the plurality of features may further include a width or a pose of the first instrument.

In some implementations, the plurality of features may further include a polygon mesh of the anatomy, a polygon mesh of the target, a size of the target, a diameter of a lumen, a volume of the anatomy, a classification of one or more anatomical regions, an anatomy type, or dimensions of one or more regions of the anatomy.

In some aspects, the anatomy may include a kidney having a plurality of poles and the location inferred for percutaneous entry includes one of the plurality of poles. In some implementations, the plurality of features may further include a kidney type, one or more dimensions of a renal pelvis, one or more dimensions of an infundibulum, or a classification of each of the plurality of poles.

In some aspects, the plan may further include a fluidics simulation of the anatomy. In some implementations, the fluidics simulation may be inferred from the one or more features based on the machine learning model.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method performed by a medical system, comprising:
receiving input data representing a mapping of an anatomy;
extracting, from the input data, a plurality of features including a position of a target within the anatomy;
inferring, from the plurality of features, a location on the anatomy for percutaneous entry based on a machine learning model trained on fluidics simulations that predict anatomical distension in response to irrigation; and
generating a plan for percutaneously accessing the target via the location inferred from the plurality of features.

2. The method of claim 1, wherein the input data includes a three-dimensional (3D) image of the anatomy.

3. The method of claim 1, wherein the input data includes a two-dimensional (2D) image of the anatomy.

4. The method of claim 1, wherein the plan includes a recommended pose of a first instrument configured for direct entry and a recommended pose of a second instrument configured for percutaneous access.

5. The method of claim 4, wherein the recommended poses of the first and second instruments are inferred from the plurality of features based on the machine learning model.

6. The method of claim 4, wherein the input data includes sensor data received from one or more sensors disposed on the first instrument.

7. The method of claim 4, wherein the plurality of features further includes a width or a pose of the first instrument.

8. The method of claim 1, wherein the plurality of features further includes a polygon mesh of the anatomy, a polygon mesh of the target, a size of the target, a diameter of a lumen, a volume of the anatomy, a classification of one or more anatomical regions, an anatomy type, or dimensions of one or more regions of the anatomy.

9. The method of claim 1, wherein the anatomy comprises a kidney having a plurality of poles and the location inferred for percutaneous entry includes one of the plurality of poles.

10. The method of claim 9, wherein the plurality of features further includes a kidney type, one or more dimensions of a renal pelvis, one or more dimensions of an infundibulum, or a classification of each of the plurality of poles.

11. The method of claim 1, wherein the plan further includes a fluidics simulation of the anatomy.

12. The method of claim 11, wherein the fluidics simulation is inferred from the one or more features based on the machine learning model.

13. A controller for a medical system comprising:
a processing system;
a memory storing instructions that, when executed by the processing system, cause the controller to:
receive input data representing a mapping of an anatomy;
extract, from the input data, a plurality of features including a position of a target within the anatomy;
infer, from the plurality of features, a location on the anatomy for percutaneous entry based on a machine learning model trained on fluidics simulations that predict anatomical distension in response to irrigation; and
generate a plan for percutaneously accessing the target via the location inferred from the plurality of features.

14. The controller of claim 13, wherein the input data includes a three-dimensional (3D) image of the anatomy or a two-dimension (2D) image of the anatomy.

15. The controller of claim 13, wherein the plan includes a recommended pose of a first instrument configured for direct entry and a recommended pose of a second instrument configured for percutaneous access, the recommended poses of the first and second instruments being inferred from the plurality of features based on the machine learning model.

16. The controller of claim 15, wherein the input data includes sensor data received from one or more sensors disposed on the first instrument.

17. The controller of claim 15, wherein the plurality of features further includes a width or a pose of the first instrument.

18. The controller of claim 13, wherein the plurality of features further includes a polygon mesh of the anatomy, a polygon mesh of the target, a size of the target, a volume of the anatomy, a classification of one or more anatomical regions, an anatomy type, or dimensions of one or more regions of the anatomy.

19. The controller of claim 13, wherein the anatomy comprises a kidney having a plurality of poles and the location inferred for percutaneous entry includes one of the plurality of poles, the plurality of features further including a kidney type, one or more dimensions of a renal pelvis, one or more dimensions of an infundibulum, a diameter of a ureter, or a classification of each of the plurality of poles.

20. The controller of claim 13, wherein the plan further includes a fluidics simulation of the anatomy, the fluidics simulation being inferred from the one or more features based on the machine learning model.
